(19) United States
(12) Patent Application Publication
(10) Pub. No.: US 2025/0079022 A1
(43) Pub. Date: Mar. 6, 2025

(54) PATIENT POPULATION FLOW MODELING

(71) Applicants: Fresenius Medical Care Holdings, Inc., Waltham, MA (US); Fresenius Medical Care AG, Bad Homburg (DE)

(72) Inventors: Alhaji Cherif, Queens, NY (US); Peter Kotanko, New York, NY (US); Nina Muellers, Bad Homburg (DE); Zdenek Cerman, Idstein (DE); Julian Dreismeier, Bad Homburg (DE); Robert Kossmann, Boston, MA (US)

(21) Appl. No.: 18/950,005

(22) Filed: Nov. 16, 2024

Related U.S. Application Data
(63) Continuation of application No. PCT/US2023/067328, filed on May 23, 2023.
(60) Provisional application No. 63/345,105, filed on May 24, 2022.

(51) Int. Cl. G16H 50/50; G16H 20/40; G16H 50/70

(52) U.S. Cl. CPC G16H 50/50; G16H 20/40; G16H 50/70

(57) ABSTRACT

Techniques for patient population flow modeling are disclosed, where such modeling can provide insights for future trends among a patient population, e.g., to facilitate allocation of resources for the patient population or to inform treatment recommendations for patients. A flow model can be used on dialysis patient populations where the patients are grouped into a compartment according to a certain criterion (or given criteria), where each compartment corresponds to the number of patients in each group, and where the number of compartments can vary depending on the given criteria. A model can then be used to describe the transition rates between the different compartments, as well as influx and efflux within each compartment. Such a model can be used, by way of example, to investigate the impact of sodium-glucose co-transporter 2 (SGLT2) inhibitors on the population dynamics of end-stage renal disease patients.

### CROSS-REFERENCE TO RELATED APPLICATIONS

This application is a bypass continuation that claims priority to International Patent Application No. PCT/US2023/067328 filed on May 23, 2023, which claims priority to U.S. Provisional Pat. App. No. 63/345,105 filed on May 24, 2022, where the entire content of each of the foregoing is hereby incorporated by reference.

### FIELD

The present disclosure generally relates to techniques for patient population flow modeling, e.g., for the efficient and effective allocation of resources to care for a patient population and/or to inform recommendations for patient treatment.

### BACKGROUND

Diabetes mellitus (DM) is common in chronic kidney disease (CKD) patients and associated with excess cardiovascular (CV) morbidity and mortality. Sodium-glucose co-transporter 2 inhibitors (SGLT2i) can have beneficial effects on morbidity and mortality of patients with DM, CV, and CKD. There remains a need for a technique to predict the impact of, inter alia, SGLT2i use on a dialysis patient census.

### SUMMARY

The present teachings include techniques for patient population flow modeling, which can provide valuable insights regarding future trends among a patient population to enable proper allocation of resources to care for the patient population and/or inform treatment recommendations. By way of example, flow modeling of population dynamics can be used on dialysis patient populations where the patients are grouped according to a given attribute or criterion (or given criteria). A flow model can be used where the patients are grouped into a compartment according to a given attribute, and where each compartment corresponds to the number of patients in each group. The number of compartments in which the patient population is grouped can vary depending on the attributes of interest. A flow model can then be used to describe the transition rates between the different compartments, as well as influx to and efflux from each compartment. Such a model can be used, by way of example, to investigate the impact of sodium-glucose co-transporter 2 (SGLT2) inhibitors on the population dynamics of end-stage renal disease patients.

In an aspect, a computer program product disclosed herein may include computer-executable code embodied in a non-transitory computer-readable medium that, when executed on one or more computing devices, performs the steps of: creating a plurality of compartments of dialysis patients from a census of dialysis patients based on one or more shared attributes from a plurality of attributes related to the dialysis patients; receiving historical data related to the census of dialysis patients and the one or more shared attributes; creating a flow model describing one or more dynamic activities between the plurality of compartments, the one or more dynamic activities including transition rates between compartments, influx into compartments, and efflux out of compartments over time, where the flow model includes a plurality of model parameters each related to the one or more dynamic activities; formatting the historical data as a set of first model parameters; inputting the set of first model parameters into the flow model; identifying a set of second model parameters, each of the second model parameters dependent on a treatment for the dialysis patients; analyzing the flow model to determine a predictive impact of the treatment on the set of second model parameters; and outputting, from the flow model, population data for the plurality of compartments where dialysis patients within one or more of the plurality of compartments are given the treatment. Aspects similarly include a method including one or more of the aforementioned steps performed by the executed code.

Implementations may include one or more of the following features. Analyzing the flow model may include determining differential predictive impacts of the second model parameters. The computer program product may further include code that, when executed on one or more computing devices, performs the step of providing an initial estimation of one or more of the second model parameters before analyzing the flow model. The one or more shared attributes may include at least one of a medical diagnosis, a treatment indication, and a current treatment. The computer program product may further include code that, when executed on one or more computing devices, performs the step of outputting, from the flow model, population data including a time dependency. The computer program product may further include code that, when executed on one or more computing devices, performs the steps of receiving updated historical data and changing one of the plurality of compartments, the plurality of attributes, and the set of first model parameters based in the updated historical data. Analyzing the flow model may include varying a treatment inclusion rate. Analyzing the flow model may include performing a sensitivity analysis of the plurality of model parameters. The sensitivity analysis may identify one or more model parameters impacted by sodium-glucose co-transporter 2 inhibitors. The treatment may include at least one of a sodium-glucose co-transporter 2 inhibitor medication and dialysis.

In an aspect, a system disclosed herein may include: a database connected to a network, the database including data related to a plurality of dialysis patients, the data including a plurality of attributes of the plurality of dialysis patients; and a processor and a memory. The memory may store computer-executable code embodied in a non-transitory computer-readable medium that, when executing by the processor, performs the steps of: creating a plurality of compartments of dialysis patients from a census of dialysis patients based on one or more shared attributes from a plurality of attributes related to the dialysis patients; receiving historical data related to the census of dialysis patients and the one or more shared attributes; creating a flow model describing one or more dynamic activities between the plurality of compartments, the one or more dynamic activities including transition rates between compartments, influx into compartments, and efflux out of compartments over time, where the flow model includes a plurality of model parameters each related to the one or more dynamic activities; formatting the historical data as a set of first model parameters; inputting the set of first model parameters into the flow model; identifying a set of second model parameters, each of the second model parameters dependent on a treatment for the dialysis patients; analyzing the flow model to determine a predictive impact of the treatment on the set of second model parameters; and outputting, from the flow model, population data for the plurality of compartments where dialysis patients within one or more of the plurality of compartments are given the treatment.

In an aspect, a method disclosed herein may include: creating a plurality of compartments of dialysis patients from a census of dialysis patients based on one or more shared attributes from a plurality of attributes related to the dialysis patients; creating a flow model featuring the plurality of compartments, the flow model identifying one or more patterns of dynamic activity between compartments; analyzing the flow model to calculate transition rates between compartments, influx into compartments, and efflux out of compartments over time; and determining, using the transition rates, influx, and efflux, a predictive impact of a treatment used by a plurality of the dialysis patients having at least one of the plurality of attributes. Aspects similarly may include a computer program product including computer-executable code embodied in a non-transitory computer-readable medium that, when executed on one or more computing devices, performs one or more of the steps of the aforementioned method.

Implementations may include one or more of the following features. The treatment may include use of sodium-glucose co-transporter 2 inhibitors. The method may further include providing a recommendation for resource allocation to one or more of the plurality of the dialysis patients having at least one of the plurality of attributes based on the predictive impact of the treatment. The method may further include predicting end-stage renal disease incidents. The method may further include determining a prevalence for a particular incident for a patient within at least one of the plurality of compartments. The particular incident may include an end-stage renal disease incident. At least a portion of the census of dialysis patients and the plurality of attributes may be included within historical data of an organization and/or synthetic data. The plurality of compartments may be created from a group of the census of dialysis patients.

In an aspect, a system disclosed herein may include: a database connected to a network, the database including data related to a plurality of dialysis patients, the data including a plurality of attributes of the plurality of dialysis patients; and a processor and a memory. The memory may store computer-executable code embodied in a non-transitory computer-readable medium that, when executing by the processor, performs the steps of: creating a plurality of compartments of dialysis patients from the plurality of dialysis patients in the database, where patients within each of the plurality of compartments include one or more shared attributes from the plurality of attributes; creating a flow model featuring the plurality of compartments, the flow model identifying one or more patterns of dynamic activity between compartments; analyzing the flow model to calculate transition rates between compartments, influx into compartments, and efflux out of compartments over time; and determining, using the transition rates, influx, and efflux, a predictive impact of a treatment used by a plurality of the dialysis patients having at least one of the plurality of attributes.

These and other features, aspects, and advantages of the present teachings will become better understood with reference to the following description, examples, and appended claims.

### BRIEF DESCRIPTION OF THE DRAWINGS

The foregoing and other objects, features and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a schematic of a flow model, in accordance with a representative embodiment.

FIG. 2 is a flow chart of a method for modeling patient populations, in accordance with a representative embodiment.

FIG. 3 shows a graph of an example of sensitivity analysis of model parameters, in accordance with a representative embodiment.

FIG. 4 includes graphs showing a simulation of an example impact of sodium-glucose co-transporter 2 (SGLT2) inhibitor at different times, in accordance with a representative embodiment.

FIG. 5 shows graphs of end-stage renal disease (ESRD) patient populations over time, in accordance with a representative embodiment.

FIG. 6 includes graphs showing model outputs simulating the impact of sodium-glucose co-transporter 2 inhibitor treatment in reduction of mortality rates and disease progression rates, in accordance with a representative embodiment.

FIG. 7 includes graphs showing model outputs simulating the impact of SGLT2 inhibitor treatment in reduction of mortality rates and progression into dialysis rates, in accordance with a representative embodiment.

FIG. 8 includes graphs showing differential impacts of model parameters, in accordance with a representative embodiment.

FIG. 9 is a flow chart of a method for modeling patient populations, in accordance with a representative embodiment.

FIG. 10 illustrates a system for patient population flow modeling, in accordance with a representative embodiment.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “about,” “approximately,” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms unless specifically stated to the contrary.

In general, the devices, systems, and methods disclosed herein relate to techniques for patient population flow modeling, and uses thereof for healthcare resource allocation, treatment, and the like. That is, patient population flow modeling as disclosed herein can provide valuable insights to relatively accurately predict future trends among a patient population for the purposes of, for example, efficient and effective allocation of resources to care for a patient population and/or providing treatment recommendations to patients. By way of example, flow modeling of population dynamics can be used on a population of dialysis patients, where the patients are grouped according to a given criteria or attribute. In some aspects, a flow model can be used, where patients are grouped into a compartment according to certain shared attributes, where each compartment corresponds to the number of patients in each group, and where the number of compartments can vary depending on the given criteria. A model can then be used to determine the transition rates between the different compartments, as well as influx into and efflux out of the compartments. Such a model can be used, by way of example, to investigate the impact of sodium-glucose co-transporter 2 (SGLT2) inhibitors on the population dynamics of dialysis patients.

SGLT2 inhibitors are a group of drugs that can have beneficial effects on the morbidity and mortality of patients with diabetes, cardiovascular (CV), and chronic kidney diseases (CKDs). SGLT2 inhibitors may reduce CV events and improve renal outcomes. Sec, e.g., “SGLT2 inhibitors: risk of diabetic ketoacidosis,” MIMS (Jun. 30, 2015) available at https://www.mims.co.uk/sglt2-inhibitors-risk-diabetic-ketoacidosis/diabetes/article/1353980, the entirety of which is incorporated by reference herein. Some examples of kidney and cardiovascular outcomes associated with SGLT2 inhibitor use are described in Annemarie B van der Aart-van der Beck, de Boer, R. A. and Heerspink, H. J., Nature Reviews, Nephrology, 18:294-206 (2022), the entirety of which is incorporated by reference herein.

Thus, it will be understood that SGLT2 inhibitors as used herein generally include a class of medicines that can lower blood sugar, e.g., in adults with type 2 diabetes. Medicines in the SGLT2 inhibitor class may include, e.g., canagliflozin, dapagliflozin, and empagliflozin, which are typically available as single-ingredient products and also in combination with other medicines such as metformin. SGLT2 inhibitors may lower blood sugar by causing the kidneys to remove sugar from the body through the urine. Using the present teachings, the impact of SGLT2 inhibitors (and/or other medications, therapies, treatments, and the like) can be ascertained in certain patient populations, where such insights can be used to efficiently and effectively treat, prescribe, distribute, etc., these same populations or other similar patient populations. Thus, a goal of the present teachings may include an ability to predict the impact of wide-spread use of SGLT2 inhibitors on CKD progression and/or the cascading effects on the evolution of the dialysis population.

The present teachings generally build on the use of a mathematical model that describes the dynamics of subjects from health through different stages of metabolic, cardiovascular, and kidney disease to dialysis. The model may describe the shift of subjects from health to dialysis. And this transition may be predicated on different and roughly known rates, which can be affected by therapy, and other factors. Thus, the present teachings may include the development of an advantageous model that provides insights into the impact of SGLT2i on dialysis population counts, where historical data (e.g., internal data from an organization) may be leveraged for the evolution of dialysis patient numbers over time.

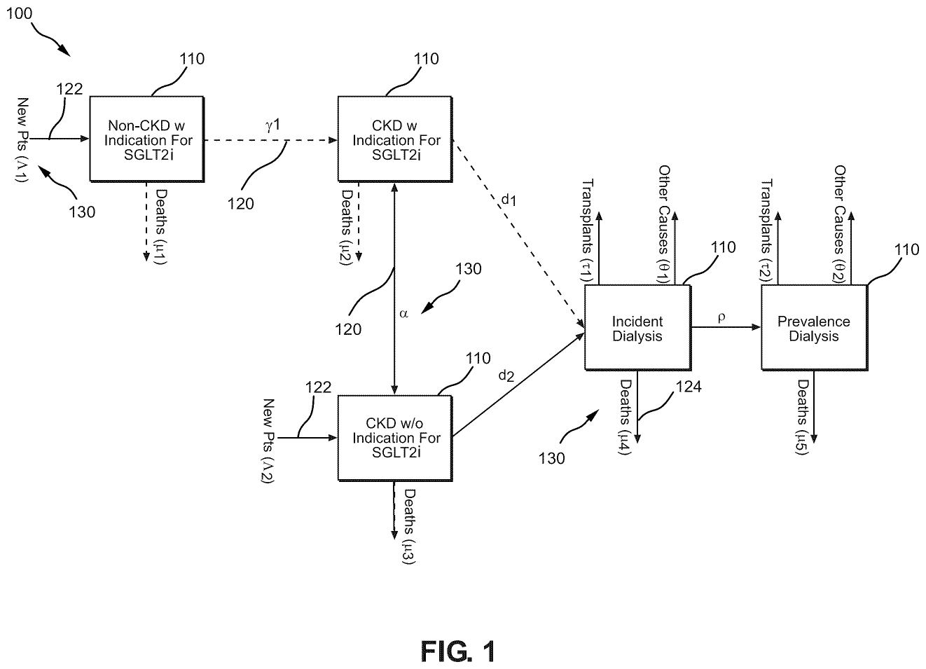

FIG. 1 shows a schematic of a flow model 100, in accordance with a representative embodiment. As discussed above, in an aspect, a patient flow model was applied to uncover insights regarding SGLT2 inhibition on the dialysis population development. Thus, the impact of SGLT2 inhibitors on the population dynamics of dialysis patients was investigated for the present teachings, and it will be understood that the description that follows describes this investigation by way of example and not limitation. One objective of the example investigation was to estimate future trends in order to properly allocate resources that can be used for the care of dialysis patients. Another objective of the example investigation was to improve decision making surrounding patient treatment and treatment recommendations. To these ends, a flow model was developed for dialysis patients, where the patients were grouped according to a plurality of compartments 110, and where each compartment corresponded to the number of patients in each group. The schematic of the flow model 100 is included in FIG. 1. In this example schematic, the population is divided into five compartments 110, namely, populations including: patients without chronic kidney disease (i.e., non-CKD) with an indication for SGLT2 inhibitors, chronic kidney disease (CKD) patients with an indication for SGLT2, CKD patients without indication for SGLT2 inhibition, incident dialysis patients, and prevalent dialysis patients. Populations of the incident dialysis and prevalent dialysis compartments may collectively be referred to herein as the total dialysis population. The model describes the transition rates 120 between different compartments, as well as influx 122 into the compartments 110 and efflux 124 out of the compartments 110.

Stated otherwise, FIG. 1 shows an example of a model schematic of patient flow with application to SGLT2 inhibitors, where the population is grouped between a general population and a dialysis population. The general population is subdivided into three categories for patients with and without indication for SGLT2 inhibitions and with and without CKD, and the dialysis population is subdivided into the incident and prevalent dialysis patients.

A mathematical model may be used that incorporates contemporary trends in a general population and a subset population of dialysis patients. The population may be divided into population groups with and without indication for SGLT2i to simplify the population demographics. For example, the population with an indication for SGLT2i may include obese and diabetic patients, while the population without indication for SGLT2i may include patients with other potential routes into CKD, e.g., genetic causes for CKD. Using these designations, the non-CKD patients with an indication for SGLT2i can either leave the compartment by death or progress into a CKD designation with an indication for SGLT2i.

The flow model may include a plurality of model parameters 130 each related to a dynamic activity, such as a transition rate 120 between compartments, influx 122 into compartments, or efflux 124 out of compartments over time. For example, in the flow model 100 shown in the figure, the model parameters 130 include: “Λ1”, representing the influx 122 of new patients into the compartment 110 “Non-CKD w Indication for SGLT2i”; “α”, representing the transition of patients between the compartment 110 “CKD w Indication for SGLT2i” and the compartment 110 “CKD w/o Indication for SGLT2i”; and “μ4”, representing the efflux 124, by death, of patients out of the compartment 110 “Incident Dialysis”. Additional model parameters 130 are indicated along the arrows of FIG. 1. In the figure, arrows pointing towards a compartment 110 indicate patient gains (e.g., influx 122), arrows pointing away indicate patient losses (e.g., efflux 124) from the compartment 110. The dashed arrows indicate model parameters 130 that are expected to be impacted by SGLT2 inhibitors. These include: “μ1”, representing the efflux, by death, of patients out of the compartment “Non-CKD w Indication for SGLT2i”; “γ1”, representing the transition of patients between the compartment “Non-CKD w Indication for SGLT2i” and the compartment “CKD w Indication for SGLT2i”; “μ2”, representing the efflux, by death, of patients out of the compartment “CKD w Indication for SGLT2i”; and “d1”, representing the transition of patients between the compartment “CKD w Indication for SGLT2i” and the compartment “Incident Dialysis”. Some of the model parameters 130 related to a transition rate 120 between compartments 110 may indicate a unidirectional transition (e.g., d1), while other model parameters 130 may indicate a bidirectional transition (e.g., α). In general, the model parameters 130 related to influx 122 into compartments 110 or efflux 124 out of compartments 110 represent a unidirectional flow of patient populations.

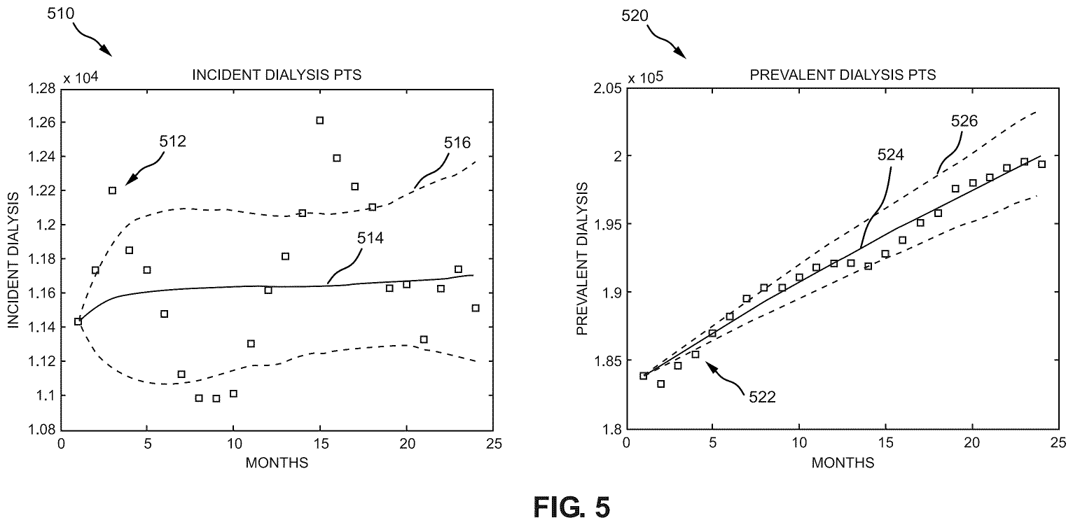

The flow model 100 may include transitions between the compartments 110 leading finally to the dialysis population. The model can be used to project end-stage renal disease (ESRD) incidents and prevalence thereof. Estimates from prior research on ESRD may be used to improve the estimates of the different rates (such as, for example, τ1—efflux out of incident dialysis, or τ2—efflux out of prevalence dialysis due to organ transplants). Using these rates, it can be shown that the model accurately describes the population dynamics of ESRD incidents and the prevalence thereof (see also discussion of FIG. 5 below).

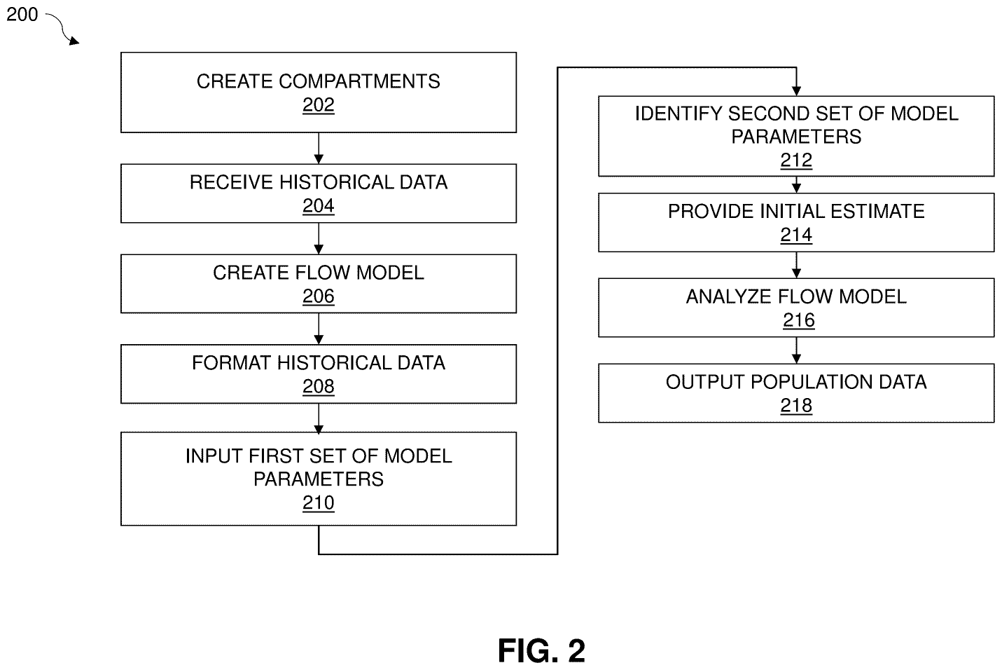

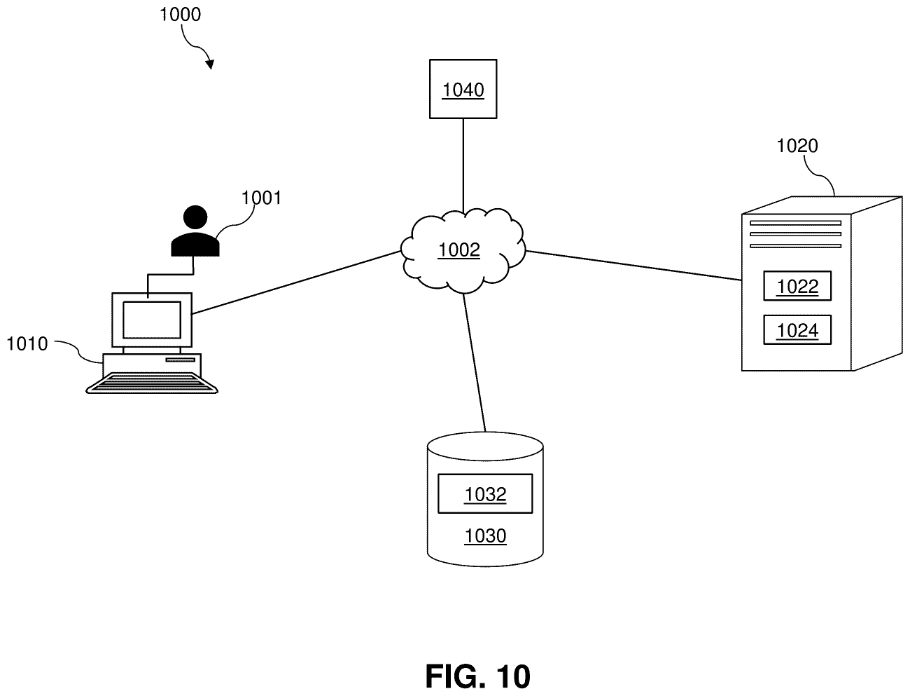

FIG. 2 is a flow chart of a method 200 for modeling patient populations, in accordance with a representative embodiment. The method 200 may utilize any of the models described herein, such as the flow model 100 described with reference to FIG. 1. The method 200 may be executed using one or more components of a system, such as the system 1000 described with reference to FIG. 10. In general, the method 200 represents a technique for analyzing patient population dynamics in response to variations in flow model parameters (where model parameters may be, for example, impacted by a specific treatment—in other words, a specific therapy applied to a patient).

As shown in step 202, the method 200 may include creating compartments, such as the plurality of compartments 110 of dialysis patients as shown in FIG. 1. The dialysis patients may be related to a plurality of attributes. The attributes may describe, for example, a medical diagnosis or condition (e.g., CKD, diabetes mellitus (DM), heart condition, etc.); a treatment indication or lack thereof (e.g., indication for SGLT2, non-indication for SGLT2); a current treatment (e.g., incident dialysis, prevalence dialysis, and so on); a biological attribute, such as age, gender, genetic factor, height, weight, other demographics, and so on; an attribute that may have implications for treatment, such as location of residence, physical ability, etc.; and others. Each compartment may be based on one or more shared attributes of patients within the compartment. In one example, each of a plurality of compartments may include a medical diagnosis and a treatment indication shared by patients within the compartment. In another example, each of a plurality of compartments may include a current treatment and a biological attribute shared by patients within the compartment. A skilled artisan will understand that the structure of the compartments, and/or the selection of shared attribute(s) that each compartment includes, may be chosen based upon the specific determination and/or prediction the artisan desires to obtain from the model.

The census that is utilized for the population used in the flow model—e.g., the census of dialysis patients or the like—may be retrieved from a database such as any of those described herein. For example, a treatment provider, medical practitioner, equipment supplier, and so on, may have information related to a plurality of patients stored in a database, where such a database (or another available database) may also include attributes thereof that can be used to compartmentalize the population as described herein. Such a database, and/or another database, may also or instead include historical data as described below.

As shown in step 204, the method 200 may include receiving historical data related to the census of dialysis patients and/or to the one or more shared attributes. For example, the historical data may relate to the prevalence of an attribute within a patient population, transition rates between compartments (e.g., the transition rates 120 of FIG. 1), and so on. The historical data may be obtained from, for example, medical or other relevant literature, internal data of an organization, and/or other sources. The historical data may be received by a computing resource of a system, such as the remote computing resource 1020 of the system 1000 of FIG. 10; and/or may be stored in a database of a system, such as the database 1030 of the system 1000.

As shown in step 206, the method 200 may include creating a flow model

describing one or more dynamic activities between the plurality of compartments (e.g., the flow model 100 describing dynamic activities between compartments 110). The dynamic activities described by the flow model may include transition rates between compartments (e.g., the transition rates 120), rates of influx into compartments (e.g., influx 122), and/or rates of efflux out of compartments over time (e.g., efflux 124). In one basic example of such a flow model, the model may include two-way transition rates between all combinations of two compartments, at least one influx into each compartment, and at least one efflux out of each compartment. Such a model would thereby account for all possible dynamic activities of patients related to the plurality of compartments. Thus, in general, the flow model may include a plurality of model parameters each related to one or more of these dynamic activities. In a more refined example of a flow model, such as the flow model 100 of FIG. 1, prior knowledge about the possible or likely flow of patients between compartments may be used in creating the flow model (for example, depending on the attributes of the compartments, it may not be possible or logical for patients to transition between all possible combinations of compartments). In some aspects, the flow model may describe the flow of patients between compartments in terms of a time dependency—for example, the flow model may describe a transition rate between two specific compartments in terms of number of patients transitioning per number of patients in population per unit time (e.g., N/1000/year). The flow model may also or instead describe other factors impacting patient populations within the compartments.

As shown in step 208, the method 200 may include formatting historical data as a set of first model parameters. This step may include one or more of: data preprocessing (such as normalization or scaling of data), extracting relevant features or variables from the raw data, data encoding (such as encoding categorical or textual data into a numerical representation), formatting the data into the appropriate input format required by the model (e.g., ensuring that the data matches the expected data type and data structure of the model), and the like. In addition, specific aspects of formatting the historical data for the flow model may vary depending on the programming language and/or framework being used for implementation of the flow model.

As shown in step 210, the method 200 may include inputting a first set of model parameters into the flow model, e.g., where the first set of model parameters is based on historical data. Depending on the specific model configuration, this step may include any of initializing the flow model, setting up the necessary configurations for the model, and model training. This step may also include passing the first set of model parameters as input arguments to one or more functions or methods that represents an input layer of the flow model. In some cases, inputting the first set of model parameters may also, or instead, include batching the data for efficient processing, handling data streaming or real-time inputs, and ensuring that the model parameters are fed to the model in the correct order. This may also or instead include using synthesized data (sec, e.g., https://en.wikipedia.org/wiki/Synthetic_data, where it should be understood that medical data may be protected and thus a “synthetic” data set is used instead).

As shown in step 212, the method 200 may include identifying a second set of model parameters. The second set of model parameters may include parameters having a dependence on a particular scenario being analyzed. For example, each of the second model parameters may be dependent on a treatment for a set of dialysis patients, such as a treatment with SGLT2i. With reference to the flow model 100 of FIG. 1, the second set of model parameters may include: γ1, representing a progression of patients into CKD; d1, representing a progression of patients into dialysis; μ1, representing mortality among patients with DM or cardiovascular disease; and μ2, representing mortality among patients with CKD. Any of these parameters may be expected to reduce or slow with the introduction of SGLT2i treatment.

As shown in step 214, the method 200 may include providing an initial estimate of one or more of the second model parameters. This step may optionally be performed depending on the specific type of model implementation (e.g., a machine learning model). An initial estimate may, for example, be derived from available data such as historical data. The second model parameters may be updated from the initial estimates as the model progresses.

As shown in step 216, the method 200 may include analyzing the flow model to determine a predictive impact of treatment on the set of second model parameters (e.g., in the case of the flow model 100, a predictive impact of treatment with SGLT2 inhibitors). This step may include, for example, analyzing the model by considering a single medical effect (such as the impact of reducing mortality rate of CKD patients, or the impact of reducing progression rate into dialysis). This step may also, or instead, include determining differential predictive impacts of the second model parameters. For example, referring to the flow model 100, impacts of SGLT2i on dialysis population counts may be differentially enhanced by reductions in mortality rate in CKD populations (second model parameter μ2)—which may have an effect of increasing the dialysis population, and progression from CKD stages into dialysis (second model parameter d1)—which may have an effect of reducing the dialysis population. Analyzing the flow model may further include varying a treatment inclusion rate. For example, varying portions of the patient population included in a compartment having an indication for SGLT2i treatment may be given the indicated treatment (e.g., 20%, 50%, 100% or other portions of the population may be given the treatment in different model scenarios). Thus, observable combinations from analyzing the flow model to determine a predictive impact of treatment may include: no change; faster/slower progression, same mortality; same progression, higher/lower mortality; or faster/slower progression, higher/lower mortality.

Analyzing the flow model may also, or instead, include performing a sensitivity analysis of the plurality of model parameters (e.g., the first set of model parameters, the second set of model parameters, or both). This approach may provide an understanding of how variations in parameter values might impact the predictions or other outputs of the model. For example, a sensitivity analysis of flow model 100 may identify one or more model parameters impacted by SGLT2 inhibitors. The sensitivity analysis may, for example, include a one-parameter sensitivity analysis, a multi-parameter sensitivity analysis, a Monte Carlo simulation, a global sensitivity analysis, or others.

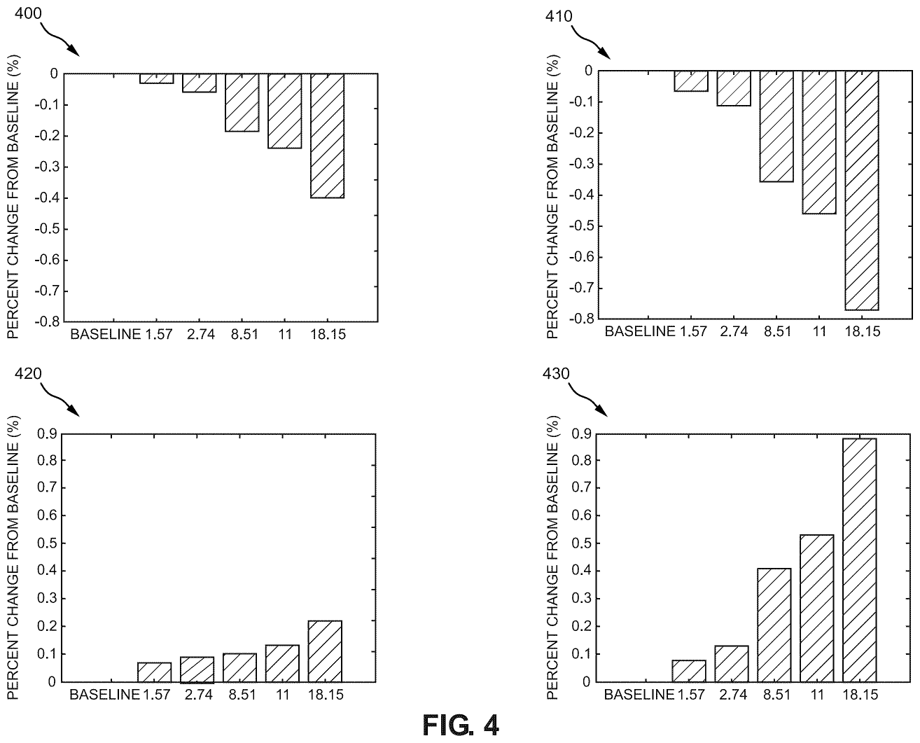

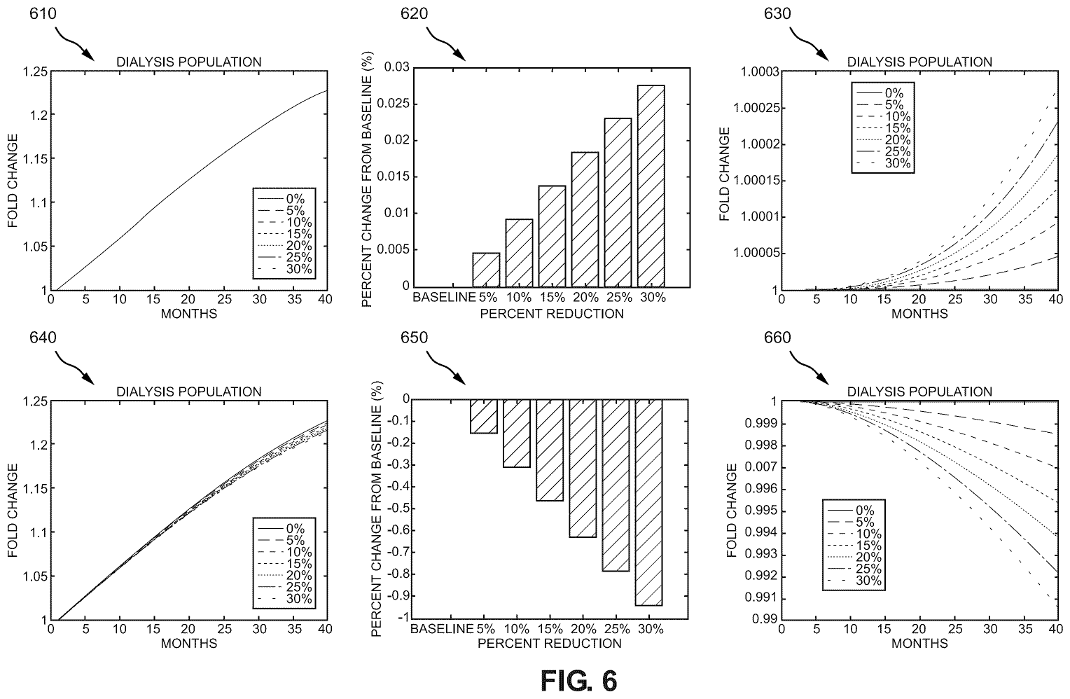

As shown in step 218, the method 200 may include outputting population data from the flow model. For example, the flow model 100 may output population data for the plurality of compartments 110. The population data output may include one or more compartments having patients (e.g., dialysis patients) that were modeled as being given a particular treatment (e.g., SGLT2i treatment). For example, scenarios may be modeled for different take-on rates (percentage of patient population in a compartment with an indication for treatment that is actually taking on the treatment). A scenario with a 0% take-on rate (no patients taking the treatment) may be useful to evaluate a baseline condition. Other scenarios may include non-zero take-on rates, e.g., up to 20% as shown and discussed in relation to FIG. 4. The population data output may include a population of each compartment after a predetermined time period (e.g., population data outputs at 1, 5, 10, and 15 years are shown in FIG. 4). In some cases, the population data output may include a time dependency—in other words, the population data may include a time dependent rate or may be a function of time. An example of population data as a function of time is shown and described in relation to FIG. 6, where graphs 630 and 660 show dialysis population over the time period 0-40 months.

In some cases, the method 200 may include iterative processing of one or more of the steps described herein. For example, after initially inputting a first set of model parameters based on historical data in step 210 (or synthetic data), a user or system may receive updated historical data in a manner similar to that described in step 204. The user or system may then perform any other steps of the method 200 again, based on the updated historical data. The method may therefore include changing one or more of the plurality of compartments, the plurality of attributes, and the set of first model parameters based in the updated historical data.

It will be understood that the method 200 may be performed using a computer program product. For example, a computer program product comprising computer-executable code embodied in a non-transitory computer-readable medium may, when executing on one or more computing devices, perform any of the steps of the method 200, including for example: creating a plurality of compartments of dialysis patients from a census of dialysis patients based on one or more shared attributes from a plurality of attributes related to the dialysis patients; receiving historical data related to the census of dialysis patients and the one or more shared attributes; creating a flow model describing one or more dynamic activities between the plurality of compartments, the one or more dynamic activities including transition rates between compartments, influx into compartments, and efflux out of compartments over time, wherein the flow model includes a plurality of model parameters each related to the one or more dynamic activities; formatting the historical data as a set of first model parameters; inputting the set of first model parameters into the flow model; identifying a set of second model parameters, each of the second model parameters dependent on a treatment for the dialysis patients; analyzing the flow model to determine a predictive impact of the treatment on the set of second model parameters; and outputting, from the flow model, population data for the plurality of compartments wherein dialysis patients within one or more of the plurality of compartments are given the treatment. It will be further understood that the method 200 may be implemented using a system, such as any as described herein. For example, such as system may include a database, a processor, and a memory, where the memory stores computer-executable code embodied in a non-transitory computer-readable medium that, when executed by the processor, performs the steps of the method 200.

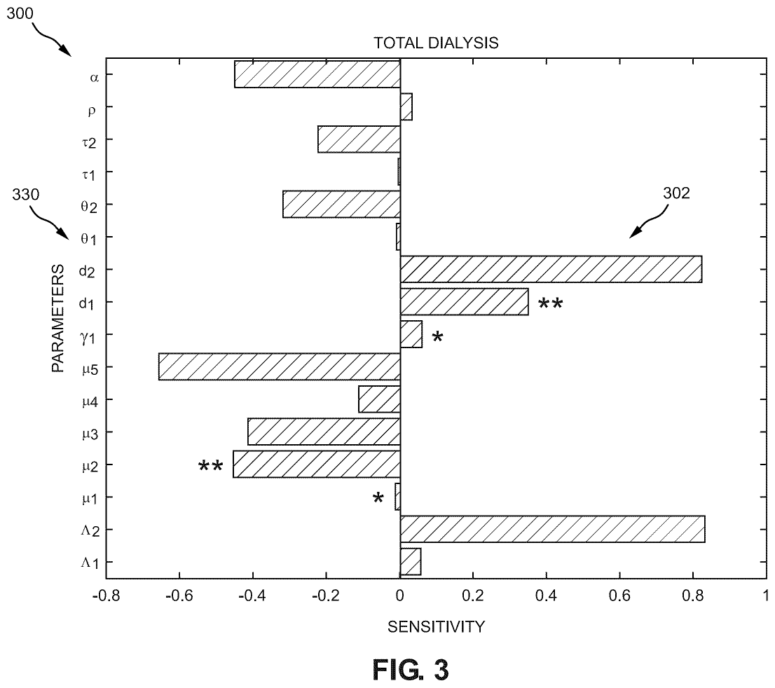

FIG. 3 shows a graph 300 of an example of sensitivity analysis of the model parameters of a flow model, e.g., the flow model 100 of FIG. 1, with respect to the total dialysis population, in accordance with a representative embodiment. Model parameters 330 therefore correspond to the model parameters 130 of FIG. 1. The stars, or asterisks, in the figure indicate the parameters impacted by SGLT2 inhibitors, specifically: d1, γ1, μ2, and μ1. Thus, as demonstrated by this figure, several sensitivity analyses were performed to determine the impact of parameter variations on the total dialysis population. As indicated by the size of the sensitivity bar 302, several parameters are predicted to have a relatively strong impact on the total dialysis population (for example, d2, d1, γ2, μ5, μ2, and Λ2). However, of the four parameters impacted by SGLT2 inhibitors, only two parameters (d1 and μ2, as indicated by a double asterisk) are expected to have a relatively strong impact on the total dialysis population. The sensitivity analysis therefor indicates that variations in the values of d1 and μ2 may have a relatively significant impact on predictions of population changes as a result of treatment with SGLT2 inhibitors.

FIG. 4 includes graphs showing a simulation of an example impact of sodium-glucose co-transporter 2 (SGLT2) inhibitors at different times, in accordance with a representative embodiment. Specifically, graph 400 shows the impact at 1 year, graph 410 shows the impact at 5 years, graph 420 shows the impact at 10 years, and graph 440 shows the impact at 15 years from the start of treatment. In each graph, the x-axis indicates the take-on rate (percentage of patient population taking on the treatment), where the baseline rate is 0% and the highest rate shown is 18.15%. The y-axis of each graph indicates the percent change from baseline in the total population of dialysis patients. As indicated in graphs 400 and 410, the dialysis population can be expected to be reduced in the short term (e.g., 1-5 years showing a negative percent change from baseline). The absolute change from baseline also increases over time in the short term (in other words, the percent reduction after 5 years, graph 410, is greater than the percent reduction after 1 year, graph 400). These results may be attributed to slowed progression from CKD stages into the dialysis population among patients receiving treatment. However, the population trend is reversed in the longer term (10-15 years), as shown in graphs 420 and 430. The percent change from baseline is shown to increase in both year 10, graph 420, and year 15, graph 430. This result may be attributed to reduced mortality within the dialysis population among patients receiving treatment (e.g., reduced efflux 124 by deaths indicated by parameters μ4 and/or μ5).

FIG. 5 shows graphs of end-stage renal disease (ESRD) patient populations over time, in accordance with a representative embodiment. Graph 510 shows data for incident dialysis patients (e.g., the patient population of the compartment 110 “Incident Dialysis” of the flow model 100 of FIG. 1) over a two year time period. Graph 520 shows data for prevalent dialysis patients (e.g., the patient population of the compartment 110 “Prevalence Dialysis” of FIG. 1) over the same time period (where Incident Dialysis patients were defined as patients initiating dialysis within the prior three months, and Prevalent Dialysis patients were defined as patients who had been receiving dialysis for greater than three months). The graphs 510 and 520 provide a comparison of a model output with historical data, where the scatter points 512 and 522 represent the historical data, the lines 514 and 524 are model output, and the dashed lines 516 and 526 represent the 95% confidence interval. The model output indicated by lines 514 and 524 illustrate an example output from the flow model (e.g., the flow model 100) of population data including a time dependency.

FIG. 6 includes graphs showing model outputs simulating the impact of sodium-glucose co-transporter 2 (SGLT2) inhibitor treatment in reduction of mortality rates and disease progression, in accordance with a representative embodiment. Several example scenarios were modeled using the flow model 100 described in FIG. 1. The flow model 100 was analyzed to determine a predictive impact of SGLT2i treatment by varying several of the model parameters 130 one at a time (in other words, in each scenario a single model parameter was varied while other model parameters were held constant). Graphs 610, 620, and 630 show outputs of modeling the impact of reduction in mortality rate (μ1 of flow model 100) of non-CKD patients with indication for SGLT2i. Each of graphs 610, 620, and 630 shows model outputs relative to a percent reduction in μ1; specifically, μ1 was reduced by 0%, 5%, 10%, 15%, 20%, 25%, and 30% in the shown simulations. The baseline condition, or 0% change to μ1, implies that no SGLT2i treatment was given. The graph 610 shows the fold change in the dialysis population relative to the initial condition. The graph 620 shows the percent change from baseline at month 40 of the simulation. The graph 630 shows the fold change in the dialysis population relative to baseline over the time period 0-40 months.

Graphs 640, 650, and 660 show outputs of modeling the impact of reducing the progression rate (γ1 of flow model 100) of non-CKD patients with indication for SGLT2i to CKD. Each of graphs 640, 650, and 660 shows model outputs relative to a percent reduction in γ1; specifically, γ1 was reduced by between 0%-30% in the shown simulations. The baseline condition—0% change to γ1—implies that no SGLT2i treatment was given. The graph 640 shows the fold change in the dialysis population relative to the initial condition. The graph 650 shows the percent change from baseline at month 40 of the simulation. The graph 660 shows the fold change in the dialysis population relative to baseline over the time period 0-40 months.

As the graphs indicate, and as was shown in the sensitivity analysis (see FIG. 3), variation in the either of model parameters μ1 or γ1 has a relatively small impact on the change in total dialysis population. Specifically, it was observed that a 30% reduction in the non-CKD mortality rate (μ1) increases the total dialysis population by 0.03%, while a similar reduction in the CKD progression rate (γ1) yields approximately 0.9% reduction in the total dialysis population.

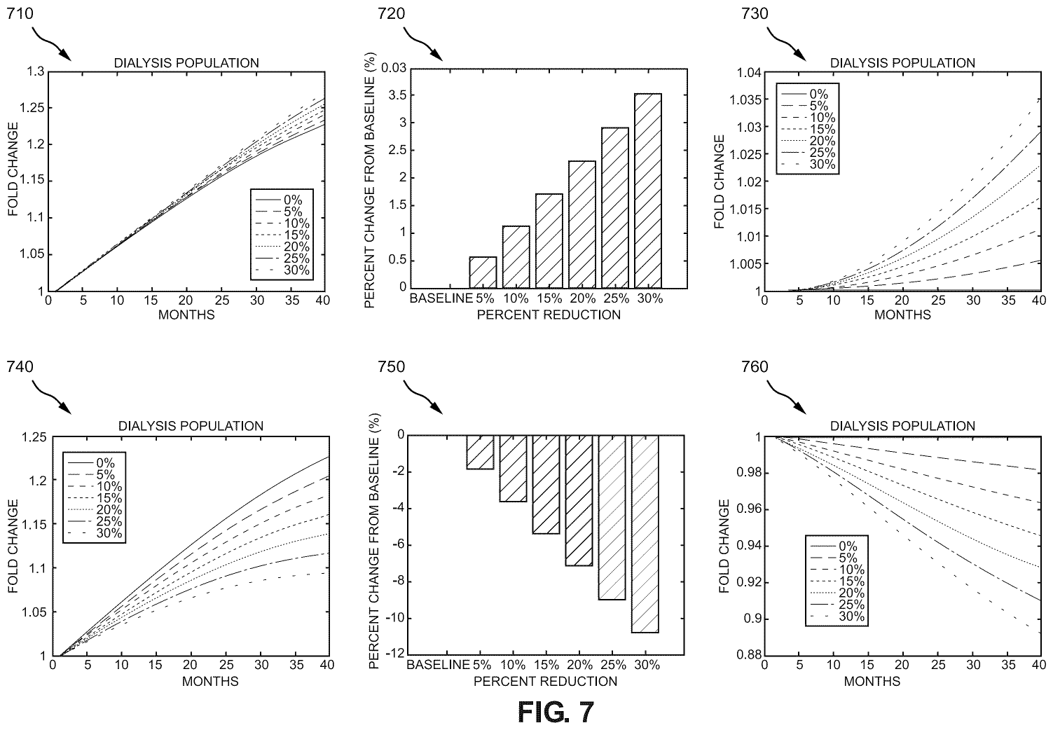

FIG. 7 includes graphs showing model outputs simulating the impact of SGLT2 inhibitor treatment in reduction of mortality rates and progression into dialysis rates, in accordance with a representative embodiment. Several example scenarios were modeled using the flow model 100 described in FIG. 1, and a similar analysis as that described with respect to FIG. 6 was performed. Graphs 710, 720, and 730 show outputs of modeling the impact of reduction in mortality rate (μ2 of flow model 100) of CKD patients with indication for SGLT2i. Each of the graphs 710, 720, and 730 shows model outputs relative to a reduction in μ2 of between 0-30%. The baseline condition, or 0% change to μ2, indicates that no SGLT2i treatment was given. The graph 710 shows the fold change in the dialysis population relative to the initial condition. The graph 720 shows the percent change in the dialysis population from baseline at month 40 of the simulation. The graph 730 shows the fold change in the dialysis population relative to baseline over the time period 0-40 months. As the graphs indicate, a reduction in CKD mortality rate (μ2) may be expected to increase the total dialysis population by up to 3.5% after 40 months (as the population not impacted by mortality may progress to dialysis).

Graphs 740, 750, and 760 show outputs of modeling the impact of reducing the progression rate (d1 of flow model 100) of CKD patients with indication for SGLT2i to incident dialysis. Model parameter d1 was reduced by between 0%-30% in the shown simulations. The baseline condition again implies that no SGLT2i treatment was given. The graph 740 shows the fold change in the dialysis population relative to the initial condition. The graph 750 shows the percent change in the dialysis population from baseline at month 40 of the simulation. The graph 760 shows the fold change in the dialysis population relative to baseline over the time period 0-40 months. It was observed that, a 30% reduction in progression rate d1 may be expected to reduce the total dialysis population up to 11% at 40 months. The model results of FIG. 7, which show a more significant impact on dialysis population dynamics than the results of FIG. 6, also correspond with the indications of the sensitivity analysis of FIG. 3 (showing a relatively high sensitivity to changes in parameters μ2 and d1).

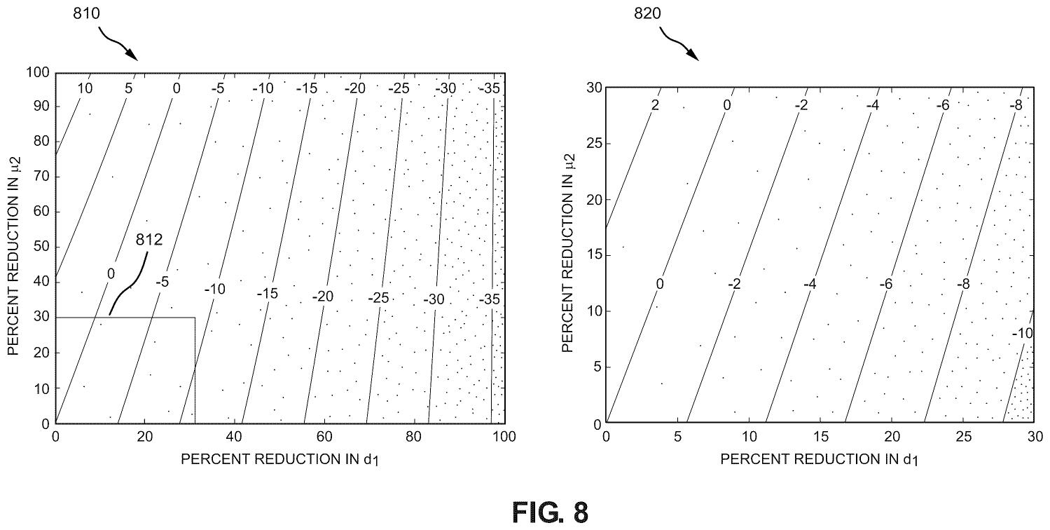

FIG. 8 includes graphs showing differential impacts of model parameters, in accordance with a representative embodiment. Specifically, a combinatorial analysis of the flow model 100 of FIG. 1 was performed, where model parameters μ2 (mortality rate of CKD patients with indication for SGLT2i) and d1 (progression rate to dialysis of CKD patients with indication for SGLT2i) were reduced over a range of 0%-100%. Graph 810 shows the percent change in dialysis population, after 40 months, as a gradient (variable stipple shading), depending on the percent reduction in d1 (x-axis) and the percent reduction in μ2 (y-axis). Graph 820 shows an enlarged view of region 812 of the graph 810, showing variation in μ2 and d1 of between 0-30%. The differential impacts of parameters μ2 (expected increase in dialysis population) and d1 (expected decrease in dialysis population), as discussed in FIG. 7, may be analyzed in greater depth using the model outputs of graphs 810 and 820. For example, a reduction in both d1 and μ2 of 30% may be expected to reduce the total dialysis population by over 8%; a reduction in both d1 and μ2 of 10% may be expected to reduce the total dialysis population by about 2%; a reduction in both d1 and μ2 of 50% may be expected to reduce the total dialysis population by about 15%; and so on.

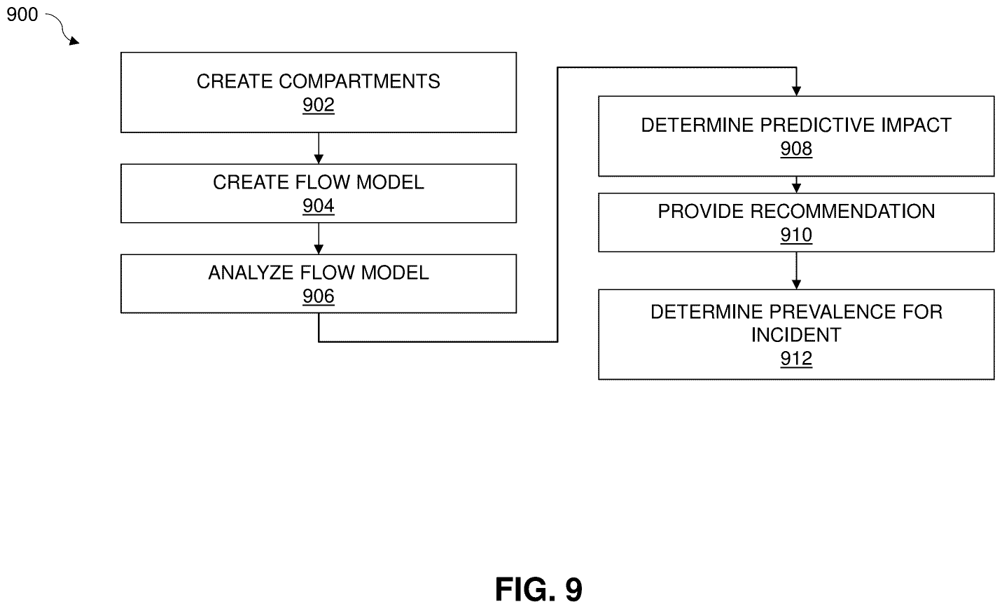

FIG. 9 is a flow chart of a method 900 for modeling patient populations, in accordance with a representative embodiment. The method 900 may utilize any of the models or techniques described herein, e.g., the flow model 100 described with reference to FIG. 1. In general, the method 900 represents a technique for analyzing patient populations using a flow model, and providing recommendations for, inter alia, healthcare resource allocation, treatment, and the like based upon the analysis.

As shown in step 902, the method 900 may include creating compartments, such as the plurality of compartments 110 of dialysis patients as shown in FIG. 1. Step 902 may be similar to step 202 of the method 200 of FIG. 2, and may thus include any of the features described with reference thereto. The plurality of compartments may be created from a group of a census of dialysis patients (e.g., a subset, or complete set, of dialysis patients undergoing treatment at a specific facility). For example, the compartments may be created from a census of dialysis patients based on one or more shared attributes from a plurality of attributes related to the dialysis patients (such as compartments including patients having a specific medical diagnosis, a treatment indication, a biological attribute, same or similar demographic information, and so on). In some cases, at least a portion of the census of dialysis patients and the plurality of attributes may be included within historical data of an organization (e.g., historical data as described with reference to step 204 of the method 200).

As shown in step 904, the method 900 may include creating a flow model featuring the plurality of compartments and identifying one or more patterns of dynamic activity between the compartments (e.g., the flow model 100 describing patterns of dynamic activity between compartments 110). Step 904 may be similar to step 206 of the method 200 of FIG. 2, and may thus include any of the features described with reference thereto.

As shown in step 906, the method 900 may include analyzing the flow model, for example, to calculate transition rates between compartments, influx into compartments, and/or efflux out of compartments over time. Step 906 may include similar analysis techniques as those described with reference to step 216 of the method 200 of FIG. 2, and may thus include any of the features described with reference thereto.

As shown in step 908, the method 900 may include determining a predictive impact, such as a predictive impact of a treatment used by a plurality of the dialysis patients. In some cases, as discussed with respect to the flow model 100 of FIG. 1, the treatment may include use of sodium-glucose co-transporter 2 (SGLT2) inhibitors. Step 908 may include similar determination techniques as those described with reference to step 216 of the method 200 of FIG. 2, and may thus include any of the features described with reference thereto. For example, determining a predictive impact may utilize one of the transition rates, influx rates, and/or efflux rates. The predictive impact may be determined for patients having a particular shared attribute or set of attributes. This step may also, or instead, include predicting specific health outcomes or incidents, such as end-stage renal disease (ESRD) incidents (this type of prediction is discussed in more detail with regards to FIGS. 6-8).

As shown in step 910, the method 900 may include providing a recommendation based on the predictive impact of the treatment (e.g., as determined in step 908). One type of recommendation may include a recommendation for resource allocation to one or more of the plurality of the dialysis patients (e.g., where a predictive impact of treatment with a SGLT2i is an increase in the dialysis population, a recommendation may include increasing resources needed for dialysis to this patient population). This step may also include acting upon the provided recommendation-in the above example, acting upon the recommendation to increase resources may include providing additional equipment, facilities, funding, etc. to the patient population of the impacted compartment. Another type of recommendation may include recommending a type of treatment, a treatment plan, or similar, to a specific patient population.

As shown in step 912, the method 900 may include determining a prevalence for a particular incident for a patient within at least one of the plurality of compartments. Referring to the flow model 100 of FIG. 1, a particular incident may include an end-stage renal disease (ESRD) incident (e.g., transition to either compartment 110 “Incident Dialysis” or “Prevalence Dialysis”). Other types of incidents may include transition to CKD, heart failure or another cardiovascular incident, stroke, and so on.

It will be understood that the method 900 may be performed using a computer program product. For example, a computer program product comprising computer-executable code embodied in a non-transitory computer-readable medium may, when executing on one or more computing devices, perform any of the steps of the method 900, including for example: creating a plurality of compartments of dialysis patients from a census of dialysis patients based on one or more shared attributes from a plurality of attributes related to the dialysis patients; creating a flow model featuring the plurality of compartments, the flow model identifying one or more patterns of dynamic activity between compartments; analyzing the flow model to calculate transition rates between compartments, influx into compartments, and efflux out of compartments over time; and determining, using the transition rates, influx, and efflux, a predictive impact of a treatment used by a plurality of the dialysis patients having at least one of the plurality of attributes. It will be further understood that the method 900 may be implemented using a system, such as any as described herein. For example, such as system may include a database, a processor, and a memory, where the memory stores computer-executable code embodied in a non-transitory computer-readable medium that, when executed by the processor, performs the steps of the method 900.

### EXAMPLE

A further description of the simulated impact of SGLT2 inhibitors on a U.S. dialysis census will now be provided, where FIGS. 1-9 may generally describe aspects of this example. Sensitivity analysis identified four SGLT2i-related parameters that affect dialysis census: mortality rate of CKD patients and CKD-to-dialysis progression exert a significant effect, while CKD mortality rates and progression to CKD have marginal impact (refer to FIGS. 1 and 3). FIGS. 6-8 illustrate the forecasted dialysis census over 40 months when CKD mortality and CKD-to-dialysis progression rates are reduced by 30%.

Table 1 below shows that a 20% SGLT2i take-on rate among CKD reduces the dialysis census from a 1.280 to a 1.269-fold change at year 5. Within ≤9 years, SGLT2i reduces the dialysis census depending on drug take-on rate. Interestingly, this trend is reversed after 10 years, resulting in rising dialysis census.

TABLE 1

| | SGLT2i Take-on rate [%] | | | |
|---|---|---|---|---|
| | 0 | 5 | 20 | 100 |
| Mortality rate | 0 | 1.65 | 6.6 | 33 |
| reduction factor | | | | |
| [%] | | | | |
| Progression rate into | 0 | 1.15 | 4.58 | 22.9 |
| dialysis factor [%] | | | | |
| Time [years] | -fold change in dialysis census relative to time 0. | | | |
| 0 | 1 | 1 | 1 | 1 |
| 1 | 1.074 | 1.073 | 1.07 | 1.052 |
| 2 | 1.151 | 1.149 | 1.142 | 1.106 |
| 5 | 1.280 | 1.277 | 1.269 | 1.215 |
| 10 | 1.291 | 1.292 | 1.294 | 1.294 |

Thus, Table 1 shows the-fold change of the dialysis census for four different SGLT2i take-on rates, with the corresponding combined effects on mortality rate in CKD and CKD-to-dialysis progression rate. For example, a 20% take-on rate corresponds to 6.6% reduction in CKD mortality rate, and 4.58% reduction in CKD-to-dialysis progression. Note that after 10 years, the dialysis census rises with SGLT2i take-on.

Thus, it appears that SGLT2i use affects dialysis census dynamically. In the first nine years, dialysis census is reduced; this phase is then followed by an increase. The ramification of time-varying SGLT2i take-on rates on dialysis census could similarly be studied using the present teachings.

As discussed herein, any of the methods and techniques described above may be performed on a system, such as a system specifically configured for patient population flow modeling. An example of such a system is described below.

FIG. 10 illustrates a system for patient population flow modeling, in accordance with a representative embodiment. In general, the system 1000 may include a networked environment where a data network 1002 interconnects a plurality of participating devices and/or users in a communicating relationship. The participating devices may, for example, include any number of user devices 1010, remote computing resources 1020, databases 1030, and other resources 1040.

The data network 1002 may be any network(s) or internetwork(s) suitable for communicating data and information among participants in the system 1000. This may include public networks such as the Internet, private networks, telecommunications networks such as the Public Switched Telephone Network or cellular networks using third generation (e.g., 3G or IMT-2000), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 1000.

Each of the participants of the data network 1002 may include a suitable network interface comprising, e.g., a network interface card, which term is used broadly herein to include any hardware (along with software, firmware, or the like to control operation of same) suitable for establishing and maintaining wired and/or wireless communications. The network interface card may include without limitation a wired Ethernet network interface card (“NIC”), a wireless 802.11 networking card, a wireless 802.11 USB device, or other hardware for wired or wireless local area networking. The network interface may also or instead include cellular network hardware, wide area wireless network hardware or any other hardware for centralized, ad hoc, peer-to-peer, or other radio communications that might be used to connect to a network and carry data. In another aspect, the network interface may include a serial or USB port to directly connect to a local computing device such as a desktop computer that, in turn, provides more general network connectivity to the data network 1002. Thus, it will be understood that any of the components of the system may include one or more communications interfaces for communicating (e.g., wired or wirelessly transmitting and/or receiving data) with other components of the system 1000.

The user devices 1010 may include any devices within the system 1000 operated by one or more users 1001 for practicing the techniques as contemplated herein. In general, a user device 1010 may include a processor 1022 and a memory 1024, where the memory 1024 stores computer-executable code embodied in a non-transitory computer-readable medium that, when executing by the processor 1022, performs one or more steps of any method or technique described herein. Thus, although these components are shown on the remote computing resource 1020 in FIG. 10, it will be understood that processing described herein may be accomplished using one or more other devices that also or instead include a processor 1022 and a memory 1024. In certain aspects, the user device 1010 includes a dialysis machine and/or a component of a dialysis system. In this manner, in some aspects, the user 1001 is one or more of a dialysis patient, a dialysis technician, medical personnel such as a doctor or nurse, and so on. In other aspects, the user device 1010 is a computing device associated with one or more of a medical professional, an insurance provider or similar, a data analyst, and the like. The user devices 1010 may also or instead include any device for managing, monitoring, or otherwise interacting with tools, platforms, and devices included in the systems and techniques contemplated herein. The user devices 1010 may be coupled to the data network 1002, e.g., for interaction with one or more other participants in the system 1000.

By way of example and not limitation, the user devices 1010 may include one or more desktop computers, laptop computers, network computers, tablets, mobile devices, portable digital assistants, messaging devices, cellular phones, smart phones, portable media or entertainment devices, or any other computing devices that can participate in the system 1000 as contemplated herein. The user devices 1010 may also or instead include any form of mobile device, such as any wireless, battery-powered device, that might be used to interact with the networked system 1000. It will also be appreciated that one of the user devices 1010 may coordinate related functions (e.g., processing some or all data, storing data, etc.) as they are performed by another entity such as one of the remote computing resources 1020 or other resources 1040.

Each user device 1010 may generally provide a user interface. The user interface may be maintained by a locally executing application on one of the user devices 1010 that receives data from, e.g., the remote computing resources 1020 or other resources 1040. In other embodiments, the user interface may be remotely served and presented on one of the user devices 1010, such as where a remote computing resource 1020 or other resource 1040 includes a web server that provides information through one or more web pages or the like that can be displayed within a web browser or similar client executing on one of the user devices 1010. The user interface may in general create a suitable visual presentation for user interaction on a display device of one of the user devices 1010, and provide for receiving any suitable form of user input including, e.g., input from a keyboard, mouse, touchpad, touch screen, hand gesture, or other user input device(s).

The remote computing resources 1020 may include, or otherwise be in communication with, a processor 1022 and a memory 1024, where the memory 1024 stores code executable by the processor 1022 to perform various techniques of the present teachings. More specifically, a remote computing resource 1020 may be coupled to the data network 1002 and accessible to the user device 1010 through the data network 1002, where the remote computing resource 1020 includes a processor 1022 and a memory 1024, where the memory 1024 stores code executable by the processor 1022 to perform the steps of a method according to the present teachings.

The remote computing resources 1020 may also or instead include data storage, a network interface (and/or other communications interface(s)), and/or other processing circuitry. In the following description, where the functions or configuration of a remote computing resource 1020 are described, this is intended to include corresponding functions or configuration (e.g., by programming) of a processor 1022 of the remote computing resource 1020, or in communication with the remote computing resource 1020. In general, the remote computing resources 1020 (or processors 1022 thereof or in communication therewith) may perform a variety of processing tasks related to patient population flow modeling as discussed herein. For example, the remote computing resources 1020 may manage information received from one or more of the user devices 1010 and/or a database 1030, and provide related supporting functions, processing one or more data sets, communicating with other resources 1040, storing data, and the like. The remote computing resources 1020 may also or instead include backend algorithms that react to actions performed by a user 1001 at one or more of the user devices 1010. The backend algorithms may also or instead be located elsewhere in the system 1000.

The remote computing resources 1020 may also or instead include a web server or similar front end that facilitates web-based access by the user devices 1010 to the capabilities of the remote computing resource 1020 or other components of the system 1000. A remote computing resource 1020 may also or instead communicate with other resources 1040 in order to obtain information for providing to a user 1001 through a user interface on the user device 1010. Where the user 1001 specifies certain criteria for data processing, this information may be used by a remote computing resource 1020 (and any associated algorithms) to access other resources 1040. Additional processing may be usefully performed in this context such as recommending certain data processing operations and techniques.

A remote computing resource 1020 may also or instead maintain, or otherwise be in communication with, a database 1030 of content such as patient data 1032 related to a plurality of dialysis patients, and/or patient data 1032 including a plurality of attributes related to a plurality of dialysis patients. The database 1030 may thus be used to store any raw and/or processed/normalized data as described herein, e.g., for use by another component in the system 1000 such as the remote computing resource 1020 and/or user device 1010. In one aspect, a remote computing resource 1020 may include a database 1030 of patient data 1032, and the remote computing resource 1020 may act as a server that provides a platform for patient population flow modeling, providing supporting services and/or output related thereto, such as services, information, and/or recommendations related to resource allocation, treatment, other logistical support, and the like.

A remote computing resource 1020 may also or instead be configured to manage access to certain content (e.g., for an enterprise associated with a user 1001 of the user device 1010). In one aspect, a remote computing resource 1020 may manage access to a component of the system 1000 by a user device 1010 according to input from a user 1001.

The other resources 1040 may include any resources that may be usefully employed in the devices, systems, and methods as described herein. For example, the other resources 1040 may include without limitation other data networks, human actors (e.g., programmers, researchers, annotators, medical personnel, insurance providers, other medical (or medical related) support service providers, analysts, and so forth), sensors (e.g., audio or visual sensors), data mining tools, computational tools, data monitoring tools, and so forth. The other resources 1040 may also or instead include any other software or hardware resources that may be usefully employed in the networked applications as contemplated herein. For example, the other resources 1040 may include payment processing servers or platforms used to authorize payment for access, content or feature purchases, or otherwise. In another aspect, the other resources 1040 may include certificate servers or other security resources for third-party verification of identity, encryption or decryption of data, and so forth. In another aspect, the other resources 1040 may include a desktop computer or the like co-located (e.g., on the same local area network with, or directly coupled to through a serial or USB cable) with one of the user devices 1010 and/or remote computing resources 1020. In this case, the other resource 1040 may provide supplemental functions for the user device 1010 and/or remote computing resource 1020. Other resources 1040 may also or instead include supplemental resources such as scanners, cameras, printers, input devices, medical equipment, and so forth.

The other resources 1040 may also or instead include one or more web servers that provide web-based access to and from any of the other participants in the system 1000. While depicted as a separate network entity, it will be readily appreciated that the other resources 1040 (e.g., a web server) may also or instead be logically and/or physically associated with one of the other devices described herein, and may, for example, include or provide a user interface for web access to a remote computing resource 1020 or a database 1030 in a manner that permits user interaction through the data network 1002, e.g., from a user device 1010.

It will be understood that the participants in the system 1000 may include any hardware or software to perform various functions as described herein. For example, one or more of the user device 1010 and the other resources 1040 may include a memory 1024 and a processor 1022.

The various components of the networked system 1000 described above may be arranged and configured to support the techniques described herein in a variety of ways. For example, in one aspect, a system 1000 includes a database 1030 connected to a network 1002, the database 1030 including patient data 1032 related to a plurality of dialysis patients, the data including a plurality of attributes of the plurality of dialysis patients; and a processor 1022 and a memory 1024. The memory 1024 may store computer-executable code embodied in a non-transitory computer-readable medium that, when executing by the processor 1022, performs the steps of: creating a plurality of compartments of dialysis patients from a census of dialysis patients (e.g., in the patient data 1032) based on one or more shared attributes from a plurality of attributes related to the dialysis patients, where the attributes may also or instead be included in the patient data 1032; receiving historical data (e.g., in the patient data 1032) related to the census of dialysis patients and the one or more shared attributes; creating a flow model describing one or more dynamic activities between the plurality of compartments, the one or more dynamic activities including transition rates between compartments, influx into compartments, and efflux out of compartments over time, where the flow model includes a plurality of model parameters each related to the one or more dynamic activities; formatting the historical data as a set of first model parameters; inputting the set of first model parameters into the flow model; identifying a set of second model parameters, each of the second model parameters dependent on a treatment for the dialysis patients; analyzing the flow model to determine a predictive impact of the treatment on the set of second model parameters; and outputting, from the flow model, population data for the plurality of compartments where dialysis patients within one or more of the plurality of compartments are given the treatment.

In another aspect, a system 1000 may include a database 1030 connected to a network 1002, the database 1030 including patient data 1032 related to a plurality of dialysis patients (or similar), the data including a plurality of attributes of the plurality of dialysis patients; and a processor 1022 and a memory 1024. The memory 1024 may store computer-executable code embodied in a non-transitory computer-readable medium that, when executing by the processor 1022, performs the steps of: creating a plurality of compartments of dialysis patients from the plurality of dialysis patients in the database 1030, wherein patients within each of the plurality of compartments include one or more shared attributes from the plurality of attributes; creating a flow model featuring the plurality of compartments, the flow model identifying one or more patterns of dynamic activity between compartments; analyzing the flow model to calculate transition rates between compartments, influx into compartments, and efflux out of compartments over time; and determining, using the transition rates, influx, and efflux, a predictive impact of a treatment used by a plurality of the dialysis patients having at least one of the plurality of attributes.

The above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application-specific integrated circuits, programmable gate arrays, programmable array logic components, or any other device or devices that may be configured to process electronic signals. It will further be appreciated that a realization of the processes or devices described above may include computer-executable code created using a structured programming language such as C, an object-oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in a number of ways. At the same time, processing may be distributed across devices such as the various systems described above, or all of the functionalities may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random-access memory associated with a processor), or a storage device such as a disk drive, flash memory, or any other optical, electromagnetic, magnetic, infrared, or other device or combination of devices. In another aspect, any of the systems and methods described above may be embodied in any suitable transmission or propagation medium carrying computer-executable code and/or any inputs or outputs from the same.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings.

Unless the context clearly requires otherwise, throughout the description, the words “comprise,” “comprising,” “include,” “including,” and the like are to be construed in an inclusive sense as opposed to an exclusive or exhaustive sense; that is to say, in a sense of “including, but not limited to.” Additionally, the words “herein,” “hereunder,” “above,” “below,” and words of similar import refer to this application as a whole and not to any particular portions of this application.

It will be appreciated that the devices, systems, and methods described above are set forth by way of example and not of limitation. For example, regarding the methods provided above, absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computing) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A computer program product comprising computer-executable code embodied in a non-transitory computer-readable medium that, when executed on one or more computing devices, performs the steps of:
creating a plurality of compartments of dialysis patients from a census of dialysis patients based on one or more shared attributes from a plurality of attributes related to the dialysis patients;
receiving historical data related to the census of dialysis patients and the one or more shared attributes;
creating a flow model describing one or more dynamic activities between the plurality of compartments, the one or more dynamic activities including transition rates between compartments, influx into compartments, and efflux out of compartments over time, wherein the flow model includes a plurality of model parameters each related to the one or more dynamic activities;
formatting the historical data as a set of first model parameters;
inputting the set of first model parameters into the flow model;
identifying a set of second model parameters, each of the second model parameters dependent on a treatment for the dialysis patients;
analyzing the flow model to determine a predictive impact of the treatment on the set of second model parameters; and
outputting, from the flow model, population data for the plurality of compartments wherein dialysis patients within one or more of the plurality of compartments are given the treatment.

2. The computer program product of claim 1, wherein analyzing the flow model includes determining differential predictive impacts of the second model parameters.

3. The computer program product of claim 1, further comprising code that, when executed on one or more computing devices, performs the step of providing an initial estimation of one or more of the second model parameters before analyzing the flow model.

4. The computer program product of claim 1, wherein the one or more shared attributes include at least one of a medical diagnosis, a treatment indication, and a current treatment.

5. The computer program product of claim 1, further comprising code that, when executed on one or more computing devices, performs the step of outputting, from the flow model, population data including a time dependency.

6. The computer program product of claim 1, further comprising code that, when executed on one or more computing devices, performs the steps of receiving updated historical data and changing one of the plurality of compartments, the plurality of attributes, and the set of first model parameters based in the updated historical data.

7. The computer program product of claim 1, wherein analyzing the flow model includes varying a treatment inclusion rate.

8. The computer program product of claim 1, wherein analyzing the flow model includes performing a sensitivity analysis of the plurality of model parameters.

9. The computer program product of claim 8, wherein the sensitivity analysis identifies one or more model parameters impacted by sodium-glucose co-transporter 2 inhibitors.

10. The computer program product of claim 1, wherein the treatment includes at least one of a sodium-glucose co-transporter 2 inhibitor medication and dialysis.

11. A method, comprising:
creating a plurality of compartments of dialysis patients from a census of dialysis patients based on one or more shared attributes from a plurality of attributes related to the dialysis patients;
receiving historical data related to the census of dialysis patients and the one or more shared attributes;
creating a flow model describing one or more dynamic activities between the plurality of compartments, the one or more dynamic activities including transition rates between compartments, influx into compartments, and efflux out of compartments over time, wherein the flow model includes a plurality of model parameters each related to the one or more dynamic activities;
formatting the historical data as a set of first model parameters;
inputting the set of first model parameters into the flow model;
identifying a set of second model parameters, each of the second model parameters dependent on a treatment for the dialysis patients;
analyzing the flow model to determine a predictive impact of the treatment on the set of second model parameters; and
outputting, from the flow model, population data for the plurality of compartments wherein dialysis patients within one or more of the plurality of compartments are given the treatment.

12. The method of claim 11, wherein analyzing the flow model includes determining differential predictive impacts of the second model parameters.

13. The method of claim 11, further comprising, before analyzing the flow model, providing an initial estimation of one or more of the second model parameters.

14. The method of claim 11, wherein the one or more shared attributes include at least one of a medical diagnosis, a treatment indication, and a current treatment.

15. The method of claim 11, further comprising outputting, from the flow model, population data including a time dependency.

16. The method of claim 11, further comprising receiving updated historical data and changing one of the plurality of compartments, the plurality of attributes, and the set of first model parameters based in the updated historical data.

17. The method of claim 11, wherein analyzing the flow model includes varying a treatment inclusion rate.

18. The method of claim 11, wherein analyzing the flow model includes performing a sensitivity analysis of the plurality of model parameters.

19. The method of claim 11, wherein the treatment includes at least one of a sodium-glucose co-transporter 2 inhibitor medication and dialysis.

20. A system, comprising:
a database connected to a network, the database including data related to a plurality of dialysis patients, the data including a plurality of attributes of the plurality of dialysis patients; and
a processor and a memory, the memory storing computer-executable code embodied in a non-transitory computer-readable medium that, when executing by the processor, performs the steps of:
creating a plurality of compartments of dialysis patients from a census of dialysis patients based on one or more shared attributes from a plurality of attributes related to the dialysis patients;
receiving historical data related to the census of dialysis patients and the one or more shared attributes;
creating a flow model describing one or more dynamic activities between the plurality of compartments, the one or more dynamic activities including transition rates between compartments, influx into compartments, and efflux out of compartments over time, wherein the flow model includes a plurality of model parameters each related to the one or more dynamic activities;
formatting the historical data as a set of first model parameters;
inputting the set of first model parameters into the flow model;
identifying a set of second model parameters, each of the second model parameters dependent on a treatment for the dialysis patients;
analyzing the flow model to determine a predictive impact of the treatment on the set of second model parameters; and
outputting, from the flow model, population data for the plurality of compartments wherein dialysis patients within one or more of the plurality of compartments are given the treatment.
